(19) United States
(12) Patent Application Publication
(10) Pub. No.: US 2020/0405136 A1
(43) Pub. Date: Dec. 31, 2020

(54) ENDOSCOPE

(71) Applicant: FUJIFILM Corporation, Tokyo (JP)

(72) Inventor: Toshio Sakamoto, Kanagawa (JP)

(73) Assignee: FUJIFILM Corporation, Tokyo (JP)

(21) Appl. No.: 17/016,647

(22) Filed: Sep. 10, 2020

Related U.S. Application Data
(63) Continuation of application No. PCT/JP2019/003792, filed on Feb. 4, 2019.

(30) Foreign Application Priority Data
Mar. 28, 2018 (JP) 2018-061913

(51) Int. Cl. A61B 1/05; G02B 7/02; G02B 23/24; A61B 1/00

(52) U.S. Cl. CPC A61B 1/05; G02B 7/02; A61B 1/00167; A61B 1/00096; G02B 23/243

(57) ABSTRACT

It is an object to provide an endoscope having high chemical resistance and easy wiring. The endoscope includes: an imaging device provided at a distal end portion of an insertion part to be inserted into a subject. The imaging device includes a solid-state imaging element having an image receiving surface which is disposed to intersect with a longitudinal direction of the insertion part and photoelectrically converting an optical image formed on the image receiving surface, a cable bundle including one or more coaxial cables respectively connected to one or more connection terminals provided on a surface of the solid-state imaging element opposite to the image receiving surface, and a collective shield wire that covers an outer periphery of the one or more coaxial cables, and a metallic pipe-shaped member into which the cable bundle is inserted. The pipe-shaped member has a cutout portion on at least a part of a peripheral surface thereof, and at an end portion of the cable bundle on the solid-state imaging element side, at least one of each shield wire of the one or more coaxial cables or the collective shield wire is joined to the pipe-shaped member by at least one of solder, metal braze, or metal paste in the cutout portion of the pipe-shaped member.

### CROSS-REFERENCE TO RELATED APPLICATIONS

This application is a Continuation of PCT International Application No. PCT/JP2019/003792 filed on Feb. 4, 2019, which claims priority under 35 U.S.C. § 119(a) to Japanese Patent Application No. 2018-061913 filed on Mar. 28, 2018. The above application is hereby expressly incorporated by reference, in its entirety, into the present application.

### BACKGROUND OF THE INVENTION

### 1. Field of the Invention

The present invention relates to an endoscope.

### 2. Description of the Related Art

An imaging device mounted on a distal end portion of an insertion part of an endoscope generally comprises a solid-state imaging element (image sensor) and a circuit board on which the solid-state imaging element is mounted, and a plurality of cables inserted through the insertion part are connected to the circuit board.

In addition, a configuration in which a cable is directly connected to a solid-state imaging element without using a circuit board has also been proposed. This makes it possible to further reduce a size of the distal end portion and reduce the number of parts (cost reduction).

For example, JP2017-046854A discloses an endoscope comprising: a lens unit that houses a plurality of lenses in a lens support member; an imaging element having an imaging surface covered by an element cover glass; a resin for adhesion that fixes the lens unit in which optical axes of the plurality of lenses are aligned with the center of the imaging surface and the element cover glass; a distal end portion whose maximum outer diameter is in a range of a finite diameter to 1.8 nm, which corresponds to a diameter of a circumscribed circle of a substrate of the imaging element; a mold portion that covers and fixes at least a part of the lens unit and the imaging element with a mold resin; and a tubular sheath that is formed to have the same outer diameter as the distal end portion and covers at least a part of the mold portion so as to be connected to the mold portion.

JP2017-046854A discloses that a plurality of transmission cables are connected to a surface of the imaging element opposite to the imaging surface, and the transmission cables are covered and fixed with the mold resin.

### SUMMARY OF THE INVENTION

An endoscope needs to be disinfected and/or sterilized using a chemical after use.

However, in the configuration in which the transmission cable is covered and fixed with the mold resin as in the endoscope of JP2017-046854A, the mold resin is vulnerable to chemicals, and thus, repeated disinfection and/or sterilization may cause deterioration of the mold resin due to chemicals and damage to the transmission cable. Further, in a case where the mold resin deteriorates, the transmission cable cannot be fixed with the mold resin. Therefore, in a case where the insertion part is bent, the transmission cable may be pulled and a load may be applied to a solder portion at an end portion of the cable, resulting in disconnection.

In addition, in the configuration in which the transmission cable is covered and fixed with the mold resin, there is a problem that it is complicated to connect shield wires of the plurality of transmission cables to each other.

An object of the present invention is to solve such a problem and to provide an endoscope having high chemical resistance and easy wiring.

The present invention solves the problems by the following configurations.

[1] An endoscope comprising:

an imaging device provided at a distal end portion of an insertion part to be inserted into a subject,

in which the imaging device includes
a solid-state imaging element having an image receiving surface which is disposed to intersect with a longitudinal direction of the insertion part and photoelectrically converting an optical image formed on the image receiving surface,
a cable bundle including one or more coaxial cables respectively connected to one or more connection terminals provided on a surface of the solid-state imaging element opposite to the image receiving surface, and a collective shield wire that covers an outer periphery of the one or more coaxial cables, and
a metallic pipe-shaped member into which the cable bundle is inserted,

the pipe-shaped member has a cutout portion on at least a part of a peripheral surface thereof, and

at an end portion of the cable bundle on the solid-state imaging element side, at least one of each shield wire of the one or more coaxial cables or the collective shield wire is joined to the pipe-shaped member by at least one of solder, metal braze, or metal paste in the cutout portion of the pipe-shaped member.

[2] The endoscope according to [1],

in which at the end portion of the cable bundle on the solid-state imaging element side, at least two of each shield wire of the one or more coaxial cables and the collective shield wire are joined to the pipe-shaped member by at least one of solder, metal braze, or metal paste in the cutout portion of the pipe-shaped member.

[3] The endoscope according to [1] or [2], further comprising:

a case member that encloses the solid-state imaging element,

in which the pipe-shaped member is joined to the case member.

[4] The endoscope according to [3],

in which the pipe-shaped member is joined to the case member by one or more of joining using at least one of solder, metal braze, or metal paste, welding, and pressure welding.

[5] The endoscope according to [3] or [4], further comprising:

a lens barrel disposed on the image receiving surface side of the solid-state imaging element,

in which the lens barrel is joined to the case member.

[6] The endoscope according to [5],

in which the lens barrel is joined to the case member via a sensor holder that holds the lens barrel.

[7] The endoscope according to [5] or [6],

in which the lens barrel or the sensor holder is joined to the case member by one or more of joining using at least one of solder, metal braze, or metal paste, welding, and pressure welding.

[8] The endoscope according to any one of [1] to [3], further comprising:

a case member that encloses the solid-state imaging element,

in which the pipe-shaped member and the case member are integrally formed.

[9] The endoscope according to any one of [3] to [8],

in which the case member encloses at least a part of the pipe-shaped member, and has an opening portion corresponding to at least at a position the cutout portion of the pipe-shaped member.

[10] The endoscope according to any one of [1] to [9],

in which the pipe-shaped member has an opening through which the cable bundle is inserted.

According to the present invention, it is possible to provide an endoscope having high chemical resistance and easy wiring.

### BRIEF DESCRIPTION OF THE DRAWINGS

FIG. 1 is a view conceptually showing an example of an endoscope system using an endoscope of the present invention.

FIG. 2 is a front view conceptually showing an example of the endoscope of the present invention.

FIG. 3 is a side view of the endoscope shown in FIG. 2.

FIG. 4 is a rear view of the endoscope shown in FIG. 2.

FIG. 5 is a cross-sectional view taken along line b-b of FIG. 2.

FIG. 6 is a cross-sectional view taken along line c-c of FIG. 2.

FIG. 7 is a perspective view showing a part of the endoscope shown in FIG. 2.

FIG. 8 is a view in which a conductive joining member of FIG. 7 is not shown.

FIG. 9 is a perspective view schematically showing another example of a pipe-shaped member.

FIG. 10 is a view for illustrating an example of a method of manufacturing an endoscope.

FIG. 11 is a view for illustrating the example of the method of manufacturing the endoscope.

FIG. 12 is a view for illustrating the example of the method of manufacturing the endoscope.

FIG. 13 is a view for illustrating the example of the method of manufacturing the endoscope.

FIG. 14 is a view for illustrating the example of the method of manufacturing the endoscope.

### DESCRIPTION OF THE PREFERRED EMBODIMENTS

Hereinafter, embodiments of an endoscope of the present invention will be described with reference to the drawings.

Description of constituents described below may be made based on a typical embodiment of the present invention, but the present invention is not limited to such an embodiment. In the drawings of the present specification, the scale of each part is appropriately changed and shown in order to facilitate visual recognition.

In the present specification, a numerical range represented by “to” means a range including numerical values before and after “to” as a lower limit value and an upper limit value.

### Endoscope

The endoscope of the embodiment of the present invention comprises: an imaging device provided at a distal end portion of an insertion part to be inserted into a subject. The imaging device includes a solid-state imaging element having an image receiving surface which is disposed to intersect with a longitudinal direction of the insertion part and photoelectrically converting an optical image formed on the image receiving surface, a cable bundle including one or more coaxial cables respectively connected to one or more connection terminals provided on a surface of the solid-state imaging element opposite to the image receiving surface, and a collective shield wire that covers an outer periphery of the one or more coaxial cables, and a metallic pipe-shaped member into which the cable bundle is inserted. The pipe-shaped member has a cutout portion on at least a part of a peripheral surface thereof, and at an end portion of the cable bundle on the solid-state imaging element side, at least one of each shield wire of the one or more coaxial cables or the collective shield wire is joined to the pipe-shaped member by at least one of solder, metal braze, or metal paste in the cutout portion of the pipe-shaped member.

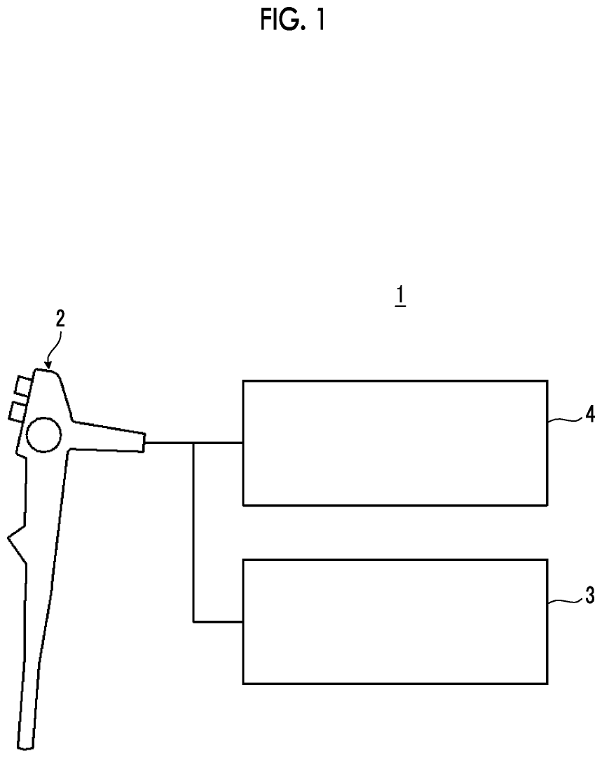

FIG. 1 conceptually shows an example of the endoscope of the embodiment of the present invention.

An endoscope system 1 comprises an endoscope 2, a light source unit 3, and a processor unit 4. The endoscope 2 has the same configuration as a general endoscope except for a portion of an imaging device 10 which will be described later. The endoscope 2 has an insertion part to be inserted into a subject, an operation part connected to the insertion part, and a universal cord extending from the operation part, and the insertion part includes a distal end portion, a bending portion connected to the distal end portion, and a flexible portion connecting the bending portion and the operation part to each other.

The distal end portion is provided with an illumination optical system that emits illumination light for illuminating an observation region, an imaging device and an imaging optical system that image the observation region, and the like. The bending portion is configured to be bendable in a direction orthogonal to a longitudinal axis of the insertion part, and a bending operation of the bending portion is performed by the operation part. In addition, the flexible portion is configured to be relatively flexible so as to be deformable according to a shape of an insertion path of the insertion part.

The operation part is provided with a button for operating an imaging operation of the imaging device of the distal end portion, a knob for operating the bending operation of the bending portion, and the like. In addition, the operation part is provided with an introduction port into which a treatment tool such as an electric scalpel is introduced, and a treatment tool channel which reaches the distal end portion from the introduction port and through which a treatment tool such as forceps is inserted is provided inside the insertion part.

A terminal of the universal cord is provided with a connector, and the endoscope 2 is connected to, via the connector, the light source unit 3 that generates illumination light emitted from the illumination optical system of the distal end portion and the processor unit 4 that processes a video signal acquired by the imaging device of the distal end portion. The processor unit 4 processes the input video signal to generate video data of the observation region, and displays and records the generated video data on a monitor.

A light guide and an electric wire group (cable bundle) are housed inside the insertion part, the operation part, and the universal cord. Illumination light generated by the light source unit 3 is guided to the illumination optical system of the distal end portion via the light guide, and a signal and electric power are transmitted between the imaging device of the distal end portion and the processor unit 4 via the electric wire group.

A distal end hard portion of the endoscope 2 is provided with an imaging device 20 and a distal end portion of the treatment tool channel, and is also provided with the illumination optical system or the like that emits illumination light guided from the light source unit 3 via the light guide.

A sensor holder 23 that holds an image sensor 21 and a lens barrel 22 of the imaging device 20 is housed in a housing hole formed in the distal end hard portion made of a metal material such as stainless steel, and is fixed to the distal end hard portion. The distal end portion of the treatment tool channel and the illumination optical system are also respectively housed in the housing hole formed in the distal end hard portion, and are fixed to the distal end hard portion.

An image receiving surface 21a of the image sensor 21 held by the sensor holder 23 fixed to the distal end hard portion is disposed substantially perpendicularly to a longitudinal axis of the insertion part.

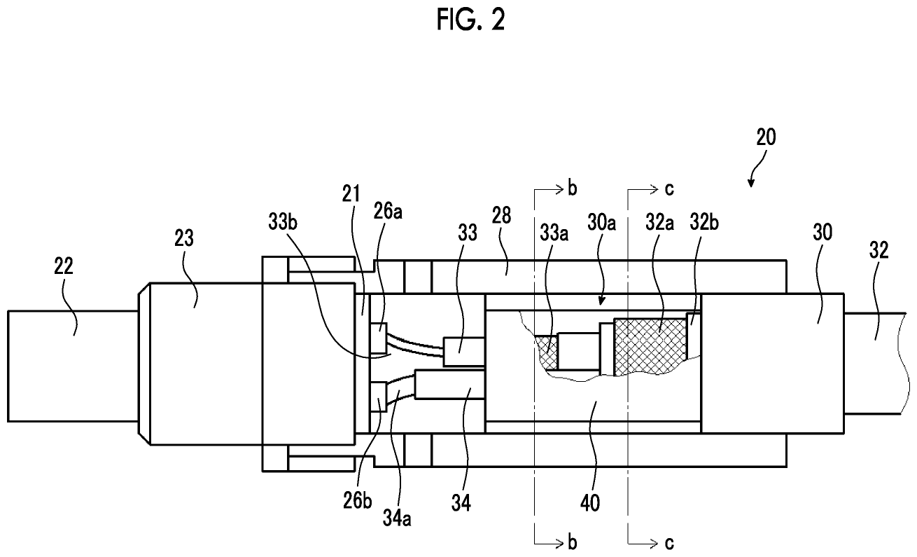

FIGS. 2 to 8 show a configuration of an imaging device mounted on a distal end hard portion of an insertion part 6 of the endoscope 2 of the embodiment of the present invention.

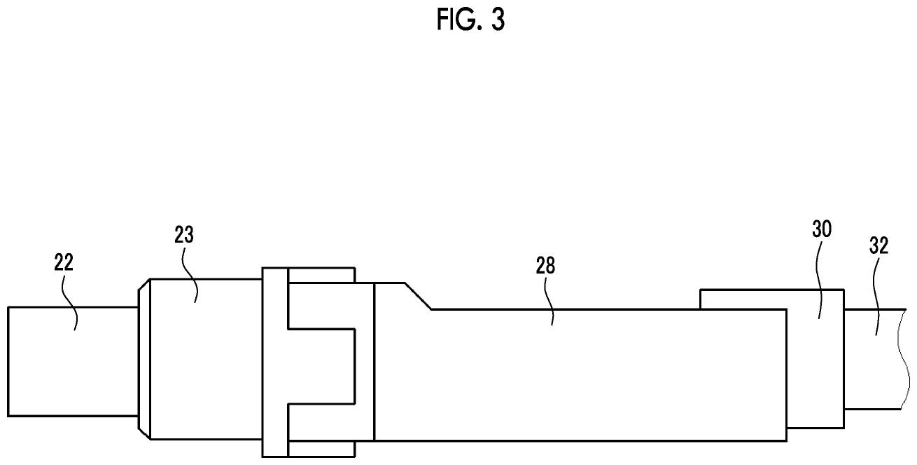

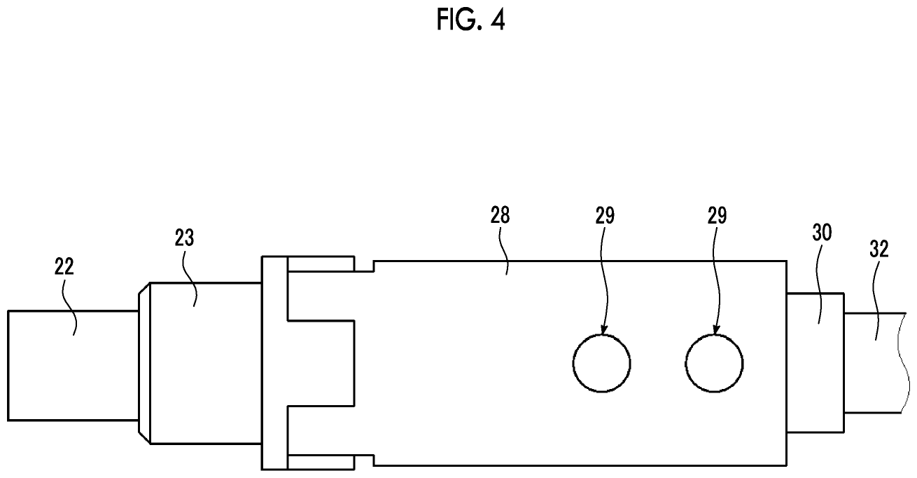

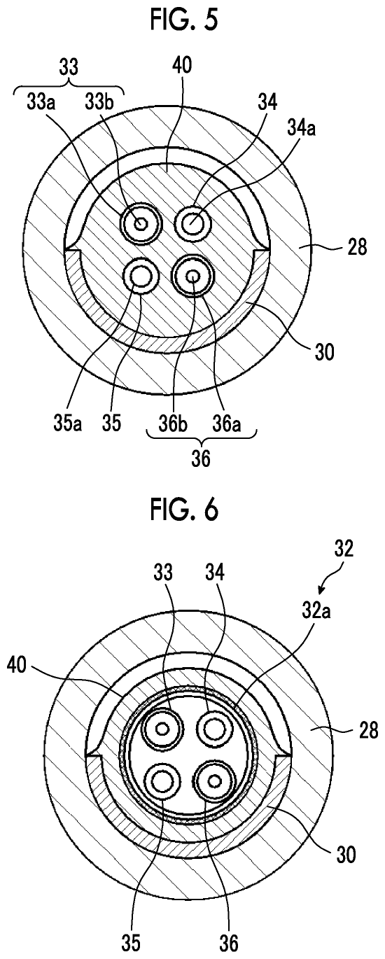

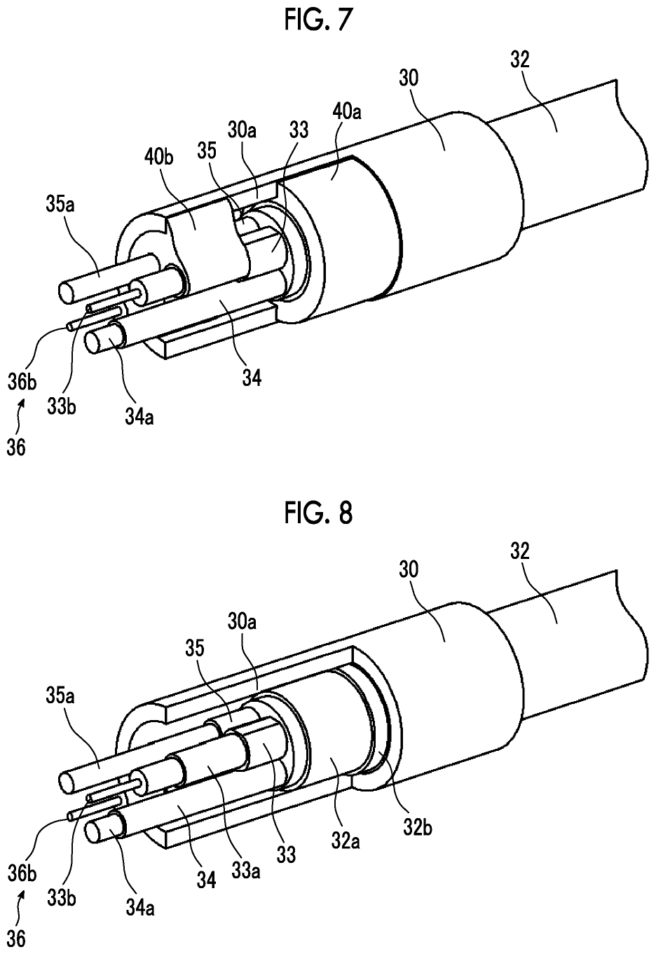

FIG. 2 is a front view conceptually showing an example of the imaging device 20 of the endoscope 2. In FIG. 2, a part of a conductive joining member 40 is not shown. FIG. 3 is a side view of the imaging device 20 shown in FIG. 2. FIG. 4 is a rear view of the imaging device 20 shown in FIG. 2. FIG. 5 is a cross-sectional view taken along line b-b of FIG. 2. FIG. 6 is a cross-sectional view taken along line c-c of FIG. 2. FIG. 7 is a perspective view showing a part of the imaging device 20. FIG. 8 is a view in which a conductive joining member in FIG. 7 is not shown.

The imaging device 20 comprises the image sensor (solid-state imaging element) 21 such as a charge coupled device (CCD) image sensor or a complementary metal oxide semiconductor (CMOS) image sensor, the lens barrel 22 that houses an imaging optical system for forming an image of a subject on the image receiving surface 21a of the image sensor 21, the sensor holder 23 that holds the image sensor 21 and the lens barrel 22, a cable bundle 32 that comprises a plurality of cables connected to a connection terminal 26 of the image sensor 21, a metallic (conductor) pipe-shaped member 30 into which the cable bundle 32 is inserted, a metallic (conductor) case member 28 that covers the image sensor 21 and a part of the pipe-shaped member 30, and the conductive joining member 40 that joins at least one of shield wires of the cables to the pipe-shaped member 30 in a cutout portion 30a of the pipe-shaped member 30. In the present invention, the conductive joining member 40 is at least one of solder, metal braze, or metal paste.

The lens barrel 22 is held by the sensor holder 23 so as to be movable along an optical axis of the imaging optical system, and the lens barrel 22 is moved such that a position of the image sensor 21 with respect to the imaging optical system is adjustable. The lens barrel 22 is fixed to the sensor holder 23 by, for example, an adhesive after the image sensor 21 is positioned.

The image sensor 21 has the image receiving surface 21a disposed to intersect with a longitudinal direction of the insertion part 6, and photoelectrically converts an optical image formed on the image receiving surface 21a. An outer diameter of the image sensor 21 as seen in a normal direction of the image receiving surface 21a is 1 mm square or less. A plurality of connection terminals for inputting and outputting a signal and electric power are provided on a back surface of the image sensor 21 opposite to the image receiving surface 21a. In FIG. 2 and the like, some (two) of the plurality of connection terminals are denoted by reference numerals 26a and 26b, and four connection terminals are provided in the examples shown in FIG. 2 and the like.

Cables included in the cable bundle 32 are electrically connected to the connection terminals, respectively.

The cable bundle 32 comprises two or more coaxial cables respectively connected to two or more connection terminals provided on a surface of the image sensor 21 opposite to the image receiving surface 21a, and a collective shield wire that covers outer peripheries of the two or more coaxial cables.

In the examples shown in FIGS. 2 to 8, the cable bundle 32 has four cables of a coaxial cable 33, a single-axis cable 34, a ground cable 35, and a coaxial cable 36, a collective shield wire 32a that covers outer peripheries of the four cables via an insulator enclosing the four cables, and a protective covering (sheath) 32b that covers an outer periphery of the collective shield wire 32a.

As shown in FIG. 2, FIG. 8, and the like, the cable bundle 32 has the sheath 32b peeled off at an end portion thereof on the image sensor 21 side to expose the collective shield wire 32a and the four cables.

In the present invention, the end portion of the cable bundle on the image sensor (solid-state imaging element) side refers to a portion where the sheath 32b is peeled off to expose the collective shield wire 32a and the cable.

The coaxial cable 33 is a coaxial cable in which a periphery of an inner conductor 33b is electromagnetically shielded by a shield wire 33a, and has the inner conductor 33b, an insulator between the inner conductor 33b and the shield wire 33a, the shield wire 33a, and a sheath that covers an outer periphery of the shield wire 33a.

As shown in FIG. 2, FIG. 8, and the like, the coaxial cable 33 has the sheath peeled off at an end portion thereof on the image sensor 21 side to expose the shield wire 33a and the inner conductor 33b.

A distal end portion of the exposed inner conductor 33b is electrically connected to the connection terminal 26a of the image sensor 21.

The coaxial cable 36 has the same configuration as the coaxial cable 33, and is electrically connected to the connection terminal of the image sensor 21 (not shown).

The single-axis cable 34 is a cable for supplying electric power to the image sensor 21, and has an inner conductor 34a and a sheath that covers an outer periphery of the inner conductor 34a.

As shown in FIG. 2, FIG. 8, and the like, the single-axis cable 34 has the sheath peeled off at an end portion thereof on the image sensor 21 side to expose the inner conductor 34a.

A distal end portion of the exposed inner conductor 34a is electrically connected to the connection terminal 26b of the image sensor 21.

The ground cable 35 is a cable for connecting the image sensor 21 to the ground, and has an inner conductor 35a and a sheath that covers an outer periphery of the inner conductor 35a.

As shown in FIG. 2, FIG. 8, and the like, the ground cable 35 has the sheath peeled off at an end portion thereof on the image sensor 21 side to expose the inner conductor 35a.

A distal end portion of the exposed inner conductor 35a is electrically connected to the connection terminal of the image sensor 21 (not shown).

The cable bundle 32 is inserted through the insertion part and the universal cord of the endoscope, and the image sensor 21 is connected to the processor unit 4 via the plurality of cables of the cable bundle 32.

The cable bundle 32 is inserted through the pipe-shaped member 30 in the vicinity of the end portion thereof.

The pipe-shaped member 30 is a metallic member into which the cable bundle 32 is inserted. The pipe-shaped member 30 has the cutout portion 30a on at least a part of a peripheral surface thereof. The pipe-shaped member 30 preferably has an opening through which the cable bundle 32 can be inserted.

In the example shown in FIG. 2, the pipe-shaped member 30 is a cylindrical member of which both ends are open, and an inner diameter thereof is a size such that the cable bundle 32 can be inserted therethrough. That is, the pipe-shaped member 30 has openings at both ends through which the cable bundle 32 can be inserted. The pipe-shaped member 30 is made of a metal having conductivity.

In addition, as shown in FIG. 2 and the like, the pipe-shaped member 30 has the cutout portion 30a on a part of the peripheral surface thereof. In a longitudinal direction of the cable bundle 32, the pipe-shaped member 30 is disposed such that the cutout portion 30a is located at a position where the collective shield wire 32a of the cable bundle 32 and the shield wire 33a of the coaxial cable 33 are exposed.

The case member 28 is a member that encloses the image sensor 21.

In the example shown in FIG. 2 and the like, the case member 28 is a hollow rectangular parallelepiped-shaped member having three open surfaces, and has a substantially U-shaped (C-shaped) cross-section perpendicular to a longitudinal direction.

The case member 28 encloses (supports) a part of the pipe-shaped member 30 that holds the cable bundle 32 and the image sensor 21, with the longitudinal direction thereof aligned with the longitudinal direction of the cable bundle 32.

It is preferable that the case member 28 encloses at least a part of the pipe-shaped member and has an opening portion at least at a position corresponding to the cutout portion of the pipe-shaped member. In the example shown in FIG. 2, the largest open surface (opening portion 28a) of the case member 28 is disposed so as to align with the cutout portion 30a side of the pipe-shaped member 30.

In addition, as shown in FIG. 4, a through-hole 29 is formed on a back surface side of the case member 28 (a surface opposite to the opening portion 28a). In a portion of the through-hole 29, the case member 28 and the pipe-shaped member 30 are joined to each other by using solder, metal braze, and metal paste (collectively referred to as a conductive joining member).

The case member 28 and the pipe-shaped member 30 may be joined to each other by a method such as welding and pressure welding. In addition, the joining position of the case member 28 and the pipe-shaped member 30 is not limited to the back surface side of the case member 28, and joining may be performed at any position as long as the case member 28 and the pipe-shaped member 30 can be joined to each other.

The image sensor 21 is disposed at one end portion of the case member 28. The case member 28 and the image sensor 21 are preferably joined to each other by an adhesive.

The sensor holder 23 is joined to an end portion of the case member 28 on the image sensor 21 side.

The case member 28 and the sensor holder can be joined to each other by a method using the above-described conductive joining member and a method such as welding and pressure welding.

Here, as shown in FIGS. 2 to 8, at the end portion of the cable bundle 32 on the image sensor 21 side, the shield wire 33a of the coaxial cable 33, the shield wire 36a of the coaxial cable 36, and the collective shield wire 32a of the cable bundle 32 are covered with the conductive joining member 40, and are electrically joined to the pipe-shaped member 30 in the cutout portion 30a of the pipe-shaped member 30.

The conductive joining member 40 is at least one of solder, metal braze, or metal paste.

As the solder, various kinds of solder used for a wiring board or the like of an endoscope in the related art are available.

As the metal braze, various kinds of metal braze used for a wiring board or the like of an endoscope in the related art are available.

As the metal paste, various kinds of metal paste such as silver paste used for a wiring board or the like of an endoscope in the related art are available.

As described above, in a configuration in which a transmission cable is covered and fixed with a mold resin in the related art, the mold resin is vulnerable to chemicals, and thus, repeated disinfection and/or sterilization may cause deterioration of the mold resin due to chemicals and damage to the transmission cable. Further, in a case where the mold resin deteriorates, the transmission cable cannot be fixed with the mold resin. Therefore, in a case where the insertion part is bent, the transmission cable may be pulled and a load may be applied to a solder portion at an end portion of the cable, resulting in disconnection.

Further, there is also a problem that it is complicated to connect shield wires of a plurality of the transmission cables to each other.

With respect to this, in the endoscope of the embodiment of the present invention, the cable bundle comprising one or more coaxial cables respectively connected to the connection terminal of the solid-state imaging element and the collective shield wire that covers the outer periphery of the one or more coaxial cables is inserted into the metallic pipe-shaped member, and at least one of each shield wire of the one or more coaxial cables or the collective shield wire is joined to the pipe-shaped member by the conductive joining member in the cutout portion of the pipe-shaped member.

Since each shield wire (collective shield wire) is joined to the pipe-shaped member by the conductive joining member, the conductive joining member does not deteriorate due to chemicals, and the shield wire and the collective shield wire can be protected.

In addition, since the conductive joining member that fixes the shield wire and the collective shield wire is at least one of solder, metal braze, or metal paste, the conductive joining member does not easily deteriorate due to chemicals. For that reason, even though the disinfection and/or sterilization is repeated, the shield wire and the collective shield wire can be maintained in a fixed state. Therefore, it is possible to suppress a problem such that a transmission cable may be pulled and a load may be applied to a solder portion at an end portion of the cable to cause disconnection, in a case where the insertion part is bent.

In addition, by joining each shield wire and the collective shield wire to each other by the conductive joining member, conduction between the shield wires can be ensured, and wiring is easy. By making potentials of a plurality of the shield wires constant, electromagnetic interference between the shield wires can be prevented, and noise can be reduced.

Here, in the example shown in FIG. 2, a configuration in which the shield wires of two coaxial cables and the collective shield wire 32a of the cable bundle 32 are all joined to the pipe-shaped member 30 in the cutout portion 30a of the pipe-shaped member 30 by the conductive joining member 40 is adopted. However, the present invention is not limited to this. It is only needed that at least one of each shield wire of a plurality of the coaxial cables or the collective shield wire of the cable bundle is joined to the pipe-shaped member by the conductive joining member in the cutout portion of the pipe-shaped member, and it is preferable that at least two of each shield wire of the plurality of coaxial cables and the collective shield wire of the cable bundle are joined to the pipe-shaped member in the cutout portion of the pipe-shaped member by the conductive joining member.

As in the example shown in FIG. 7, the inner conductor 35a of the ground cable 35 may be joined to the pipe-shaped member in the cutout portion 30a of the pipe-shaped member 30 by the conductive joining member 40.

In addition, as in the example shown in FIG. 7, a cable which is not to be joined to the conductive joining member 40 such as the single-axis cable 34 may not be joined to the conductive joining member 40.

In the example shown in FIG. 2, the conductive joining member 40 has a size for covering all of the shield wires of two coaxial cables and the collective shield wire 32a of the cable bundle 32, but as in the example shown in FIG. 7, the conductive joining member 40 may be divided into a plurality of members such as a conductive joining member 40a that has a size for covering the collective shield wire 32a of the cable bundle 32 and a conductive joining member 40b that has a size for covering the shield wire of the coaxial cable.

In the example shown in FIG. 8, a configuration in which the cable bundle 32 has four cables is adopted. However, the present invention is not limited to this as long as the cable bundle 32 has one or more coaxial cables, and the cable bundle 32 may have one to three cables or five or more cables.

In addition, in the example shown in FIG. 8, a configuration in which the cable bundle 32 has two coaxial cables is adopted. However, the present invention is not limited to this, and the cable bundle 32 may have one coaxial cable or three or more coaxial cables.

In the example shown in FIG. 2, a configuration in which the pipe-shaped member 30 has a cylindrical shape and has the cutout portion 30a on a part of the peripheral surface thereof is adopted. However, the present invention is not limited to this as long as the shield wires are connectable by the conductive joining member.

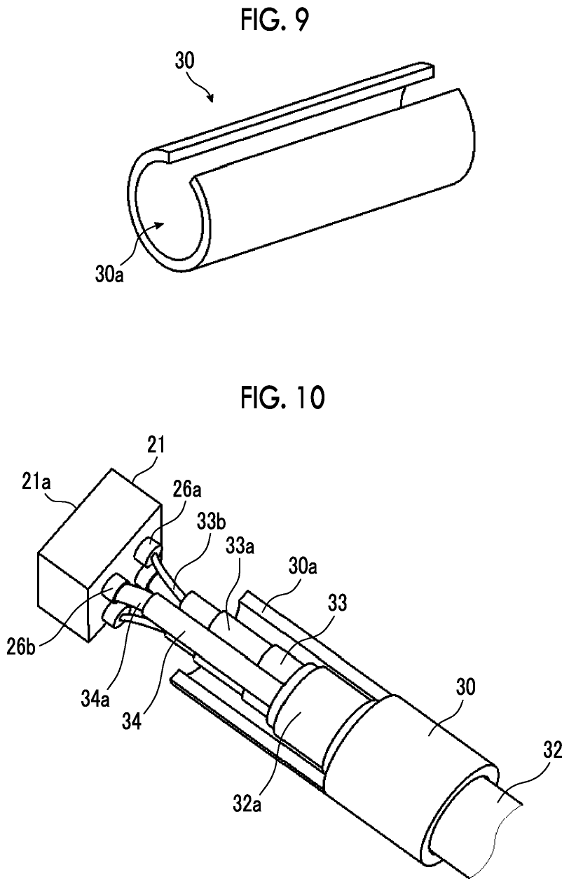

For example, as in the example shown in FIG. 9, the pipe-shaped member 30 may be a cylindrical member having a cutout portion in a shape of a slit parallel to the longitudinal direction on the peripheral surface thereof, that is, a member having a substantially C-shaped cross-section perpendicular to the longitudinal direction.

In the example shown in FIG. 2, the pipe-shaped member 30 has a cylindrical shape having an opening through which the cable bundle 32 can be inserted. However, the present invention is not limited to this as long as the cable bundle 32 can be held. For example, the pipe-shaped member may be a columnar member, or may have a shape having a slit capable of holding the cable bundle 32 on the peripheral surface thereof. In other words, the pipe-shaped member may be a member having a substantially C-shaped cross-section perpendicular to the longitudinal direction.

In the examples shown in FIG. 2 and the like, a configuration in which the pipe-shaped member 30 and the case member 28 are formed as separate members and the two members are joined to each other is adopted. However, the present invention is not limited to this, and the pipe-shaped member 30 and the case member 28 may be integrally formed.

In the examples shown in FIG. 2 and the like, a configuration in which the lens barrel 22 is joined to the case member 28 via the sensor holder 23 is adopted. However, the present invention is not limited to this, and the lens barrel 22 may be directly joined to the case member 28.

A configuration in which the shield wire of the coaxial cable and the collective shield wire of the cable bundle are covered with the conductive joining member 40, and further, the cutout portion 30a of the pipe-shaped member 30 or the opening portion 28a of the case resin mold is filled with resin mold may be adopted.

It is preferable that materials of the pipe-shaped member 30, the case member 28, and the sensor holder are those having conductivity, such as stainless steel and brass.

Next, a method of manufacturing the imaging device included in the endoscope of the embodiment of the present invention will be described with reference to FIGS. 10 to 14.

First, as shown in FIG. 10, the pipe-shaped member 30 is inserted through a distal end portion of the cable bundle 32, and the inner conductor of each cable of the cable bundle 32 is connected to the connection terminal 26 of the image sensor 21. The connection between the inner conductor and the connection terminal 26 can be performed with solder, metal braze, metal paste, or the like.

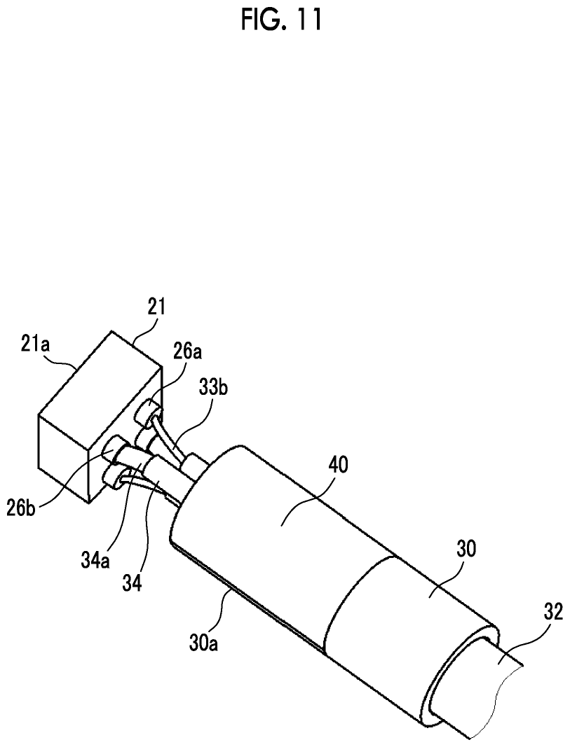

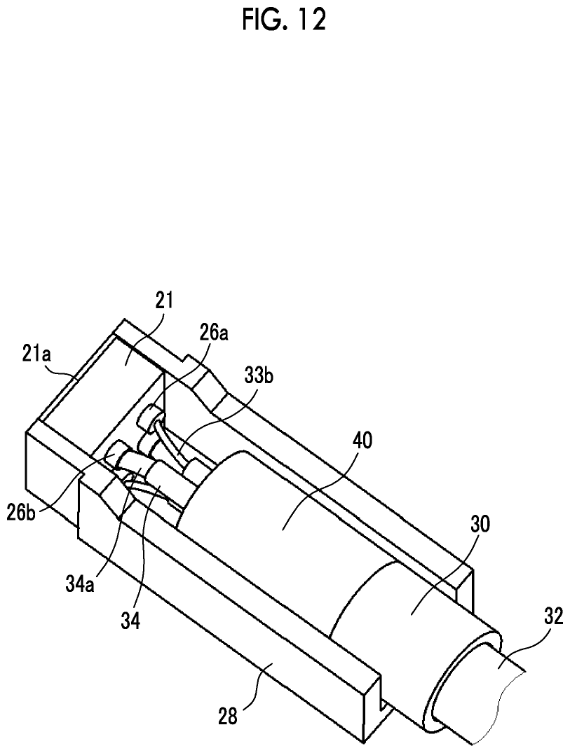

Next, as shown in FIG. 11, the shield wire of each cable and the collective shield wire 32a of the cable bundle 32 are joined to the pipe-shaped member 30 by the conductive joining member 40 in the cutout portion 30a of the pipe-shaped member 30.

As described above, since the conductive joining member 40 is any of solder, metal braze, and metal paste, the shield wire and the pipe-shaped member 30 need only be joined to each other by the conductive joining member 40 by a method according to a material to be used.

After the shield wire and the collective shield wire 32a are joined to the pipe-shaped member 30 by the conductive joining member 40 in the cutout portion 30a of the pipe-shaped member 30, the inner conductor of each cable may be connected to the connection terminal 26 of the image sensor 21.

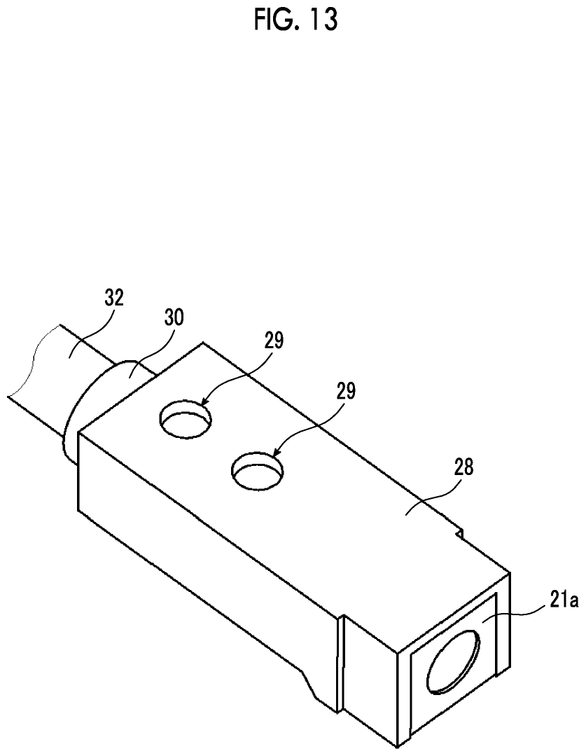

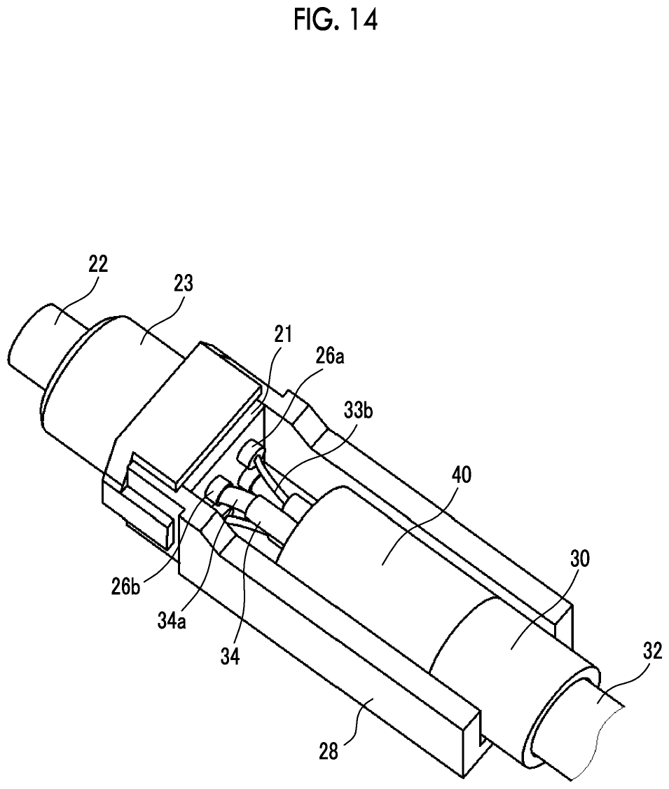

Next, as shown in FIGS. 12 and 13, the case member 28 is joined to the pipe-shaped member 30 so as to enclose a part of the pipe-shaped member 30 and the image sensor 21. In this case, it is preferable that the direction of the cutout portion 30a of the pipe-shaped member 30 is aligned with the opening portion 28a of the case member 28. In addition, it is preferable that the image receiving surface 21a of the image sensor 21 and the end surface of the case member 28 are flush with each other.

As described above, the case member 28 and the pipe-shaped member 30 can be joined by a method using a conductive joining member (solder, metal braze, and metal paste) and a method such as welding and pressure welding.

After that, as shown in FIG. 14, the sensor holder 23 holding the lens barrel 22 is joined to the end surface of the case member 28 on the image sensor 21 side.

As described above, the case member 28 and the sensor holder 23 can be joined by a method using a conductive joining member (solder, metal braze, and metal paste) and a method such as welding and pressure welding.

Thus, the imaging device of the endoscope is manufactured.

Although the endoscope of the embodiment of the present invention has been described above in detail, the present invention is not limited to the above-described embodiments, and various improvements and changes may be made without departing from the scope of the present invention.

### EXPLANATION OF REFERENCES

1: endoscope system
2: endoscope
3: light source unit
4: processor unit
5: monitor
6: insertion part
7: operation part
8: universal cord
9: connector
10: distal end portion
11: bending portion
12: flexible portion
13: introduction port
14: treatment tool channel
20: imaging device
21: solid-state imaging element (image sensor)
21a: image receiving surface
22: lens barrel
23: sensor holder
26: connection terminal
28: case member
30: pipe-shaped member
30a: cutout portion
32: cable bundle
32a: collective shield wire
32b: protective covering (sheath)
33, 36: coaxial cable
33a, 36a: shield wire
33b, 34a, 35a, 36b: inner conductor
34: single-axis cable
35: ground cable
40, 40a, 40b: conductive joining member

What is claimed is:

1. An endoscope comprising:
an imaging device provided at a distal end portion of an insertion part to be inserted into a subject,
wherein the imaging device includes
a solid-state imaging element having an image receiving surface which is disposed to intersect with a longitudinal direction of the insertion part and photoelectrically converting an optical image formed on the image receiving surface,
a cable bundle including one or more coaxial cables respectively connected to one or more connection terminals provided on a surface of the solid-state imaging element opposite to the image receiving surface, and a collective shield wire that covers an outer periphery of the one or more coaxial cables, and
a metallic pipe-shaped member into which the cable bundle is inserted,
the pipe-shaped member has a cutout portion on at least a part of a peripheral surface thereof, and
at an end portion of the cable bundle on the solid-state imaging element side, at least one of each shield wire of the one or more coaxial cables or the collective shield wire is joined to the pipe-shaped member by at least one of solder, metal braze, or metal paste in the cutout portion of the pipe-shaped member.

2. The endoscope according to claim 1,
wherein, at the end portion of the cable bundle on the solid-state imaging element side, at least two of each shield wire of the one or more coaxial cables and the collective shield wire are joined to the pipe-shaped member by at least one of solder, metal braze, or metal paste in the cutout portion of the pipe-shaped member.

3. The endoscope according to claim 1, further comprising:
a case member that encloses the solid-state imaging element,
wherein the pipe-shaped member is joined to the case member.

4. The endoscope according to claim 3,
wherein the pipe-shaped member is joined to the case member by one or more of joining using at least one of solder, metal braze, or metal paste, welding, and pressure welding.

5. The endoscope according to claim 3, further comprising:
a lens barrel disposed on the image receiving surface side of the solid-state imaging element,
wherein the lens barrel is joined to the case member.

6. The endoscope according to claim 5,
wherein the lens barrel is joined to the case member via a sensor holder that holds the lens barrel.

7. The endoscope according to claim 5,
wherein the lens barrel or the sensor holder is joined to the case member by one or more of joining using at least one of solder, metal braze, or metal paste, welding, and pressure welding.

8. The endoscope according to claim 1, further comprising:
a case member that encloses the solid-state imaging element,
wherein the pipe-shaped member and the case member are integrally formed.

9. The endoscope according to claim 3,
wherein the case member encloses at least a part of the pipe-shaped member, and has an opening portion at least at a position corresponding to the cutout portion of the pipe-shaped member.

10. The endoscope according to claim 1,
wherein the pipe-shaped member has an opening through which the cable bundle is inserted.
